(19) United States
(12) Patent Application Publication
(10) Pub. No.: US 2012/0156185 A1
(43) Pub. Date: Jun. 21, 2012

(54) NUTRITIONAL COMPOSITIONS COMPRISING CITRUS FIBERS

(75) Inventors: Anat Solomon, Gedera (IL); Haim Aviv, Rehovot (IL)

(73) Assignee: HERBAMED LTD., Rehovot (IL)

(21) Appl. No.: 13/393,277

(22) PCT Filed: Aug. 31, 2010
(86) PCT No.: PCT/IL10/00720

§ 371 Date: Feb. 29, 2012

Related U.S. Application Data
(60) Provisional application No. 61/238,251, filed on Aug. 31, 2009.

(51) Int. Cl. A61K 36/752; A61P 3/06; A61P 3/00; A61K 31/122

(52) U.S. Cl. 424/94.1; 424/736; 514/690

(57) ABSTRACT

The present invention provide edible compositions comprising soluble and insoluble dietary fibers at a ratio of about 1:1 ratio, having a palatable taste, useful for reducing the serum lipid content in a mammal, and further for reducing the blood glucose level, enhancing the feeling of satiety and keeping a desired body weight.

### FIELD OF THE INVENTION

The present invention relates to nutritional compositions comprising specific proportions of soluble to non-soluble citrus fibers effective for lowering cholesterol blood levels and enhancing feeling of satiety, having pleasant taste and texture.

### BACKGROUND OF THE INVENTION

Cholesterol is an essential lipid component in all mammalian cells. It is used to regulate the fluidity of cellular membranes and serves as a precursor for certain hormones, vitamin D and bile acids. Cholesterol is synthesized in the liver and is transported with the blood to peripheral tissues by lipoproteins. The liver has a dual function in the metabolism of cholesterol, being capable of both synthesizing cholesterol and converting surplus cholesterol into bile acids. It is also capable of excreting cholesterol into the bile.

Typically, the average person consumes between 350-400 milligrams of cholesterol daily, while the recommended intake is around 300 milligrams. Increased dietary cholesterol consumption, especially in conjunction with a diet high in saturated fat intake, can result in elevated serum cholesterol. Having an elevated serum cholesterol level is a well-established risk factor for coronary artery diseases and other cardiovascular diseases, and possibly some cancers. Therefore, there is a need to mitigate the undesired effects of cholesterol accumulation.

High cholesterol levels are generally considered to be total cholesterol levels of 200 mg/dl and above. The particular components of the lipid profile, in addition to the total level, are also of significance as risk factors. Elevated levels of low-density lipoprotein (LDL), typically above 130 mg/dl is a cause for concern, as these loosely packed lipoproteins are more likely to lodge within the cardiovascular system leading to the formation of plaques. Low levels of high-density lipoproteins (HDL) are an additional risk factor, as they serve to sweep artery-clogging cholesterol from the blood stream. The ratio of total cholesterol to HDL is considered a better indication of high cholesterol risks.

It is generally recommended to decrease serum cholesterol levels through dietary changes, a program of physical exercise, and lifestyle changes. Intake of saturated fat, particularly of animal origin, and dietary cholesterol, should be strictly limited and soluble fiber consumption should be increased. Keeping such a strict diet has been found to be efficient in reducing the serum cholesterol level; however, it is hard to maintain over prolonged periods of time.

Modifying the intestinal metabolism of lipids has been demonstrated to be effective in reducing high cholesterol levels. Hampering the absorption of triglycerides, cholesterol, bile acids or a combination thereof result in a lowering of cholesterol levels in the serum.

The most widely used lipid-lowering drugs in recent years have been statins, that reduce serum cholesterol levels by inhibiting hydroxymethylglutaryl CoA reductase (HMG CoA reductase), the first and rate-limiting enzyme of cholesterol synthesis. Bile acid resins, fibrates, nicotinic acid derivatives and various fish oil concentrates with a high content of omega.-3-fatty acids are also widely used.

A number of nutritional factors have been shown to improve serum cholesterol levels. For example, the use of phytosterols and phytostanol and their esters has been well documented in human clinical trials and in animal studies to lower serum cholesterol levels. This cholesterol lowering effect has been attributed to interference with the absorption of dietary cholesterol. Phytosterols, being structurally similar to cholesterol, competitively bind with cholesterol or sterol receptor sites, thus preventing cholesterol uptake. Unlike their cholesterol counterparts, phytosterols are very poorly absorbed, and some are not absorbed at all. Therefore, phytosterols do not contribute to an increase in serum cholesterol levels. In addition to competing for receptor sites, phytosterols also compete for the enzyme cholesterol esterase. This enzyme is required for the breakdown of cholesterol to its components, which may be absorbed through the microvilli which line the wall of the small intestine.

Flavonoids consists a major class of phytochemicals found commonly in fruits and vegetables. It has been suggested that flavonoids have a protective role against cardiovascular disease, potentially due their antioxidant, anti-platelet aggregation and anti-inflammatory effects, as well as by increasing HDL, lowering blood pressure, and improving endothelial function. Central to the pathogenesis of atherosclerosis is the oxidation of low-density lipoprotein (LDL). The chemical structure of flavonoids gives the compound free radical scavenging ability, their activity as metal chelators may contribute to the antioxidant effects (Ding, E. L. et al. 2006. Nutr. Metab. 3, 1-12).

Several studies examined the correlation between dietary intake of flavonoids and coronary heart disease. In a study of a Finish population it has been found that total flavonoid intake was significantly associated with reduction of coronary mortality in women (Knekt P et al. 1996. BMJ 312, 478-481). In addition, flavonoid intake was negatively correlated with myocardial infraction and strongly positively correlated with a decrease of mortality from heart disease in elderly man. (Hertog M et al. 1993. Lancet. 342, 1007-1111).

Plants are the source for dietary fibers, which in the plant function as structure-maintaining elements. Cellulose, hemicellulose, polysaccharides, pectins, gums, mucilages, and lignins are dietary fibers. Although these fibers are unrelated chemically, they all are resistant to breakdown by the human digestive system.

There are two major kinds of dietary fibers—insoluble (cellulose, hemicellulose, lignin) and soluble (gums, mucilages, pectins). Soluble fibers are amenable to digestion by colonic microflora present in the lower intestines. Soluble dietary fibers are believed to have a beneficial effect in the reduction of high serum cholesterol levels and reducing the risk associated with such elevated levels. It has been suggested that due to their viscosity, the soluble fibers alter the composition of the bile acid pool which result in lower cholesterol level and decrease in dietary fat absorption (Queenan K M et al. 2007. Nutr. 6, 6-14). It was also suggested that they delay the absorption of glucose into the blood stream preventing significant swings in blood sugar level. Soluble dietary fiber can have the additional beneficial effect of reduced constipation and improved regularity. Insoluble fibers are not digested by the body. They promote regularity and softening of stools. They also help to satisfy appetite by creating a full feeling.

However, too much fiber in the diet can create undesirable gastrointestinal side effects such as flatulence, diarrhea, abdominal cramps, etc. as well as weight gain due to the relatively large amount that is needed to be consumed in order to reach the health benefits.

Orange fruit, particularly the orange peel, contain dietary fiber and an array of potent antioxidants including flavonoids (hesperidin and neringenin predominantly as glycosides), carotenoids (xanthophylls, cryptoxanthins, carotenes), and vitamin C in addition to other beneficial phytochemicals such as folate. These compounds are believed to be significant contributors to the preventive effects of orange fruit against cancer and heart disease (Franke, A. A. et al. 2005 J. Agric. Food Chem. 53, 5170-5178).

Various kinds of edible compositions comprising dietary fibers and targeted at lowering blood cholesterol level have been proposed. For example, U.S. Pat. No. 5,510,337 discloses an agent for the suppression or lowering of blood lipids, cholesterol and neutral fats, which is derived from a plant and comprises, as an effective ingredient, an edible dietary fiber which contains cellulose and lignin as its main constituents and maintains the cellulose and lignin in a bonded state as in the plant.

U.S. Pat. No. 5,382,443 discloses ready-to-eat cereals containing psyllium. The cereals contain psyllium which has been extruded and pre-wetted, that is, admixed with water prior to mixing with other cereal ingredients. The cereals are useful in lowering the serum cholesterol levels of individuals who consume them.

U.S. Pat. No. 6,221,421 discloses foodstuffs and drink mixes containing extruded fiber-containing intermediates. The extruded intermediates include a soluble fiber source and an insoluble fiber source, and are useful in preparing baked goods, drink mixes, liquid drinks and other foodstuffs. Processes for preparing the intermediates and the foodstuffs and methods of lowering cholesterol with the foodstuffs are also disclosed.

U.S. Pat. No. 7,030,092 discloses an ultra-high fiber supplement that comprising guar, oat, psyllium, locust bean gum, pectin, green tea, multi-anthocyanadins, pyridoxine, and folic acid, that promotes satiety, caloric reduction, and weight loss. The supplement improves cardiovascular health and reduces cardiovascular inflammation and the risk of heart disease. Also it reduces serum lipoprotein oxidation and risk of free-radical related diseases. Additional benefits include the lowering of plasma homocysteine by the addition of folic acid and pyridoxine. Consumption of the supplement aids in reducing absorption and assimilation of ingested toxins.

U.S. Patent Application Publication No. 2008/027024 discloses dietary fiber compositions comprising effective amounts of glucomannan, xanthan gum, and alginate to produce a desired viscosity, food products comprising an effective amount of the dietary fiber composition and methods of producing same. Methods for promoting satiety, promoting weight loss, lowering blood glucose levels, or lowering blood cholesterol levels in a mammal are also disclosed.

International Application Publication. No. WO2010/038238 discloses dietary fiber compositions comprising fenugreek dietary fibers and use of these compositions as dietary supplements, functional foods or pharmaceutical preparations for promoting satiety and weight loss, lowering cholesterol, controlling blood glucose, maintaining colon health and modulating inflammation.

A dietary composition for the reduction of cholesterol levels in human blood, based on certain types of vegetable and fruit fibers, including fibers of citrus fruit has been disclosed (GB Patent No. 2,253,984). The composition was found to be also effective in reducing blood triglycerides and glucose levels. The composition was typically formulated as a nutritional bar.

There is an unmet need and it would be beneficial to have nutritional compositions effective in reducing the blood lipid content, having a desired amount of soluble fiber and palatable taste.

### SUMMARY OF THE INVENTION

The present invention provides nutritional compositions having palatable taste and texture that are effective in reducing the cholesterol blood level as well as the triglycerides and glucose blood levels. The compositions comprise soluble and insoluble citrus fibers in a beneficial ratio of about 1:1 and a unique combination of flavoring agents. The compositions of the present invention are formulated in a unit dosage form, particularly as a nutritional bar. Furthermore, the compositions of the present invention enhance the feeling of satiety, and thus contribute to body weight maintenance and prevention of obesity.

According to one aspect, the present invention provides an edible composition effective in reducing the cholesterol blood level comprising a malleable matrix comprising (a) citrus flakes at from about 5% to about 20% by weight of the total weight of the composition, wherein the ratio of the soluble to insoluble fibers is from 1:1.5 to 1.5:1.0; and (b) a flavoring agent selected from the group consisting of sesame seeds, sesame paste, chocolate, coconut, banana, dates and any combination thereof; wherein the composition is in a unit dosage form.

As used herein, the term “citrus flakes” refers to dried flakes (5-15% moisture, typically 5-10% moisture) of crushed orange fruit, before or after juice is squeezed.

The citrus flakes of the present invention are characterized in high fiber content, wherein the ratio of soluble to insoluble fibers is from about 1:1.5 to 1.5:1, typically about 1.25:1 to 1.25:1, more typically about 1:1. The citrus flakes further comprise polyphenol antioxidants, particularly flavonoids. According to certain embodiments, the citrus flakes comprise polyphenols at from 5% to 10% by weight of the total weight of the flakes. According to other embodiments, the edible citrus flakes comprise flavonoids at from 2% to 6% by weight of the total weight of the flakes. According to typical embodiments, hesperidin consists about 80-90% of the total flavonoids.

Flavonoids from other sources as well as additional antioxidants can also be included in the compositions of the present invention. According to typical embodiments, the additional antioxidant is CoQ10.

According to additional embodiments, the composition further comprises polyunsaturated fats selected from the group consisting of omega-3 fatty acid and omega-6 fatty acid. According to one embodiment, the polyunsaturated fat is from a fish origin. According to other embodiments, the polyunsaturated fat is from a plant origin.

The present invention now discloses that residue obtained after squeezing the juice from citrus fruit is highly suitable as a fiber source due to its beneficial ratio of soluble to insoluble fibers. The presence of additional beneficial component such as flavonoids as described above is an additional advantage of citrus fibers. However, the citrus flakes posses a bitter and tannin-like astringent taste that is the main disadvantage of hitherto known citrus-fiber containing nutritional compositions, particularly nutritional bars.

The present invention now discloses that adding at least one of several natural flavoring agents selected from the group consisting of sesame, sesame paste (also referred to as tahini, tahine or tahina), chocolate, coconut, banana, dates or combinations thereof, significantly improves the taste of the composition. Sesame and sesame paste were found to particularly improve the composition taste and texture. In addition, sesame and sesame paste are highly suitable for use with the compositions of the present invention, as they increase the composition's nutritional value. Sesame seeds contain about 25% protein and are particularly rich in the essential amino acids methionine and tryptophan often lacking in adequate quantities in many plant proteins. The fat in sesame seeds comprises mostly (82%) unsaturated fatty acids, including 38% monounsaturated and 44% polyunsaturated fatty acids. Importantly, sesame seeds and paste contain high amounts of available iron, and thus, according to theses embodiments, the composition of the present invention is also effective as an iron source. Alternatively or additionally, dates or products thereof (including date honey and date paste) are added to mask the bitterness of the citrus flakes. Date fruit serve as an excellent natural source for sugar, containing in the mesocarp about 70%-80%. In addition to their use as sweeteners, date fruit and products thereof are known to contain active anti-oxidants (particularly flavonoids) and significant amounts of bioavailable iron and potassium.

Thus, according to certain embodiments, the flavoring agent is selected form the group consisting of sesame, sesame paste and a combination thereof. According to typical embodiments, the composition comprises sesame seeds at from about 3% to about 11% by weight of the total weight of the composition and/or sesame paste at from about 7% to about 15% by weight of the total weight of the composition.

According to other embodiments, the flavoring agent is selected from date fruit, date paste, date honey and any combination thereof. According to typical embodiments, the composition comprises date fruit and/or products thereof at a concentration of from about 10% to about 40%, typically from about 20%-35%.

According to other embodiments, the composition further comprises a carbohydrate selected from the group consisting of fructose, sorbitol, xylitol, glucose, sucrose, dextrose and any combination thereof. In certain embodiments, the carbohydrate is selected as to be compatible for use by diabetic subjects.

Optionally, comminuted fruit is added to the composition of the present invention essentially to enhance its structure. As an example, apple puree may be used for this purpose.

According to additional embodiments, the composition of the present invention further comprises additional dietary ingredients. According to one embodiment, the dietary ingredient is selected from the group consisting of minerals, vitamins, amino acids and hormones, including phytohormones. Vitamins and minerals may be added in accordance with the limits provided by health authorities. According to certain embodiments, a composition according to the present invention comprises all recommended vitamins and minerals. The vitamins typically include A, B1, B2, B12, folic acid, niacin, panthotenic acid, biotin, C, D, E and K. The minerals typically include iron, zinc, iodine, copper, manganese, chromium and selenium. Electrolytes, such as sodium, potassium and chlorides, trace elements and other conventional additives may also be added in recommended amounts.

According to some embodiments, the composition comprises phytosterols at a concentration of from about 0.5% to about 2.0% by weight of the total weight of the composition. According to certain typical embodiments, the composition comprises phytosterols mixture derived from pine trees containing total sterols ≧99.0%, including cholesterol ≦1%, Beta-sitosterol 70-80%, beta-sitostanol ≦15%, Campesterol ≦15%, Stigmasterol ≦2%, Campestanol ≦5% and other sterols ≦3%.

According to additional embodiments, the composition further comprise formulation agents selected from the group consisting of fillers, stabilizers, diluents, binders, buffers, lubricants, preservatives, emulsifiers, coating agents, suspending agents and surface active compounds. According to typical embodiments, the preservative is selected from the group consisting of propylene glycol/glycerol and citric acid. Citric acid is further beneficial as it is also act as anti-oxidant.

The composition of the present invention may be formulated in any unit dosage form suitable for oral consumption. According to typical embodiments, the unit dosage form is a nutritional or health bar. According to typical embodiments, the bar is of from about 20 to about 60 g. According to certain currently typical embodiments, the bar weight is from 25-50 g. According to further embodiments, the bar is packed in hermetically sealing wrapping.

The compositions of the present invention are effective in reducing the serum total cholesterol level and/or the serum LDL-cholesterol levels and/or serum triglyceride levels and/or in increasing the HDL/LDL cholesterol ratio.

Without wishing to be bound by any specific theory or mechanism of action, the composition of the present invention may influence the serum cholesterol level by inhibiting the activity of hydroxymethylglutaryl (HMG) CoA reductase, the first and rate-limiting enzyme of cholesterol synthesis.

According to an additional aspect, the present invention provides a method for reducing serum lipid concentration comprising orally administering to a subject in need thereof a therapeutically effective amount of the composition of the present invention. According to one embodiment, the composition is effective in reducing the serum total cholesterol level. According to other embodiments, the composition is effective in reducing the serum triglyceride level. According to further embodiments, the composition is effective in reducing the LDL-cholesterol level or in enhancing the HDL to LDL ratio.

According to this aspect of the invention, the term “therapeutically effective amount” refers to the amount capable of reducing the serum lipid concentration, particularly the concentration of cholesterol, HDL and LDL by at least 5%, typically by at least 10%, further typically by at least 15%, more typically by 20%-25% and more.

The administration regime of the composition of the present invention will depend on parameters associated with the subject serum lipid level as well as on characteristics of the treated individual (age, weight, gender, etc.) as is known to a person skilled in the art.

According to typical embodiments, a daily quantity of from about 50 g to about 100 g of the composition of the present invention is consumed by the subject. This quantity provides about 2 g to 8 g of each of soluble and insoluble fibers. With a unit form, e.g. nutritional bar, of about 50 g, a daily intake of two units is required, which is an easy to follow and convenient regime.

Consumption of the composition of the present invention also enhances the feeling of satiety, and thus contributes to the maintenance of a desired body weight, prevents obesity, and lowers the subject's levels of blood cholesterol and glucose.

Other objects, features and advantages of the present invention will become clear from the following description.

### BRIEF DESCRIPTION OF THE FIGURES

FIG. 1A shows an RP-LC chromatogram of the flavonoid fraction from orange dry flakes derived form Valencia variety fruit. Absorbance at 285 nm is shown. Hesperidin, the major flavonoid in orange peel, is shown at t_{R}=12.395 min. FIG. 1B shows the structure of hesperidin.

FIG. 2 demonstrates the antioxidant capacity of 10 μM of hesperidin (FIG. 2A) compared to 10 μM of Trolox (FIG. 2B). Tᵢₙₕ, represents the antioxidant capacity. Tᵢₙₕ for hesperidin and trolox were 62.5 and 23.5 min respectively. Data were obtained from ORAC assay.

FIG. 3 show the correlation between antioxidant capacity as expressed in ORAC assay vs. concentration of total methanolic extract of orange material (FIG. 3A) and hesperidin concentration (FIG. 3B). The correlations were calculated by linear regression analysis. Higher R² coefficient was observed for hesperidin compared to the total extract.

FIG. 4 shows the change in blood cholesterol levels of 116 subjects during the clinical trial. All participants consumed 2 bars daily for 3-6 months. Black bars: beginning of the trial; dotted bars: end of treatment

FIG. 5 shows the blood cholesterol levels of 51 male and 62 female subjects at the beginning and at the end of the clinical trial. All participants consumed 2 bars daily for 3-6 months.

FIG. 6 shows the change in the HDL to LDL ratio during the clinical trial period. 113 subjects participated in this trial. Each person consumed 2 bars daily for 4 months. Black bars: beginning of the trial; dotted bars: end of treatment

### DETAILED DESCRIPTION OF THE INVENTION

The present invention relates to palatable edible compositions having high nutritional value, effective in reducing the serum cholesterol levels.

The compositions of the present invention comprise fruit fibers, particularly citrus fibers in the beneficial ratio of about 1:1 of soluble to insoluble fibers and at least one flavoring agent selected from the group consisting of sesame seeds, sesame paste, chocolate, coconut, banana and dates. In additional embodiments, other flavoring agents can be added, including, but not limiting to, nuts, peanut, peanut butter, raisins, apricot, peach, cranberries and other berries such as blueberries. In addition to the favorable ratio of soluble to insoluble fibers, the compositions of the present invention overcome the shortcomings of the citrus bitter taste characterizing hitherto known compositions, using flavoring agents that further contribute to the texture and nutritional value of the composition, particularly providing high amounts of iron readily available for absorbance and effective amounts of vitamins and anti-oxidants.

Fruit Fibers

“Dietary fibers” is a collective term for a variety of plant substances that are resistant to digestion by human gastrointestinal enzymes. Dietary fibers can be classified in two major groups depending on their solubility in water. In humans, matrix (or structural) fibers, including lignins, cellulose, and some hemicelluloses, are insoluble; natural gel-forming fibers, including pectin, gums, mucilage and the reminder of hemicelluloses are soluble. Soluble dietary fibers are mostly made up of complex carbohydrates, for example fructo- or galactooligosaccharides, β-glucans etc. The physiological activity of dietary fibers is affected by their physical and chemical characteristics, including water holding capacity (WHC), cation exchange capacity, bile acid binding capacity and fermentation capability, and varies with composition, content, binding state, cooking condition and source of the fibers. Soluble fibers have been shown to lower total and LDL cholesterol levels; however, the degree of cholesterol reduction caused by soluble fibers varies significantly, depending on the fiber source. Moreover, the amount of soluble fibers reported to lower the cholesterol level is too high to be consumed in the diet on a daily basis.

The compositions of the present invention are advantageous over hitherto known fiber-containing compositions for lowering serum cholesterol level: the composition comprises soluble and insoluble fibers at about 1:1 ratio by weight, which is now disclosed to be highly effective in exerting the cholesterol reduction activity of the fibers. Without wishing to be bound by any specific theory or mechanism of action, the efficiency of this ratio may be attributed to the soluble fiber fraction being active in cholesterol reduction, while the insoluble fraction having a function in prolonging the time in which the soluble fraction is maintained in the intestine. The cholesterol decrease is attributed to the decreased absorption of the bile acid due to the presence of soluble fibers, which result in the removal of steroids from the body by fecal excretion that in turn cause an increase in the cholesterol catabolism and increase in the secretion of bile acids, a decrease in lipoprotein cholesterol secretion and reduction in the total body pool of cholesterol.

In addition to the beneficial effect on the serum lipid profile and as anti-oxidants, the fibers present in the composition of the present invention enhance the feeling of satiety. As indicated above, soluble fiber is generally resistant to human digestive enzymes, except for colonic microflora present in the lower intestines, and thus, they reside in the intestine for relatively prolonged periods of time. Soluble fibers are also known for their high water and ion-binding capacity. The water absorbance is the major factor contributing to the feeling of satiety and delay of hunger. This feeling reduces the overall energy intake by the subject, leading to at least controlling if not reducing the body weight. Keeping an adequate body weight and BMI values, in addition of being a target for itself, is a major factor in preventing hypercholesterolemia and high blood glucose levels particularly in individuals susceptible to develop such disorders.

According to typical embodiments the fibers used according to the teachings of the present invention are derived from citrus. Typically, a commercial product sold as “citrus fiber” is used. This product can be obtained from crushed orange fruit including the pulp, skin and juice, or may be the remaining residue after the citrus fruit are squeezed in the juice industry. Such product contain about 55-65 weight % of dietary fibers.

Fiber's Active Ingredients

The citrus origin for fibers is further advantageous over other sources as it contains significant amounts of bioflavonoids (Lin, L. Z. and Harnly, J. M. J. 2007. Agric. Food. Chem. 55, 1084-1096).

Flavonoids display a large range of structures and they are responsible for the major organoleptic characteristics of plant-derived food and beverages, particularly color and taste properties. Flavonoids also contribute to the nutritional quality of fruit and vegetables. As typical for phenolic compounds, flavonoids, can act as potent antioxidants and metal chelators. They also have been recognized to have anti-inflammatory, anti-allergic, hepatoprotective, antithrombotic, anti-atherosclerotic, antiviral, and anti-carcinogenic activities. Oxidative modification of low-density lipoprotein (LDL) by free radicals is an early event in the pathogenesis of atherosclerosis. The rapid uptake of oxidative-modified LDL via a scavenger receptor leads to the formation of deleterious foam cells, while flavonoids may delay LDL oxidation due to their antioxidant properties. The antioxidative capacity of flavonoids is a result of their high propensity to transfer electrons, chelate ferrous ions, and scavenge reactive oxygen or nitrogen species (ROS/NOS) by acting as a chain breaking antioxidants. Furthermore, a Japanese study reported an inverse correlation between flavonoid intake and total plasma cholesterol concentrations (Arai Y et al. 2000 J Nutr 130, 2243-2250). Manthey has shown that the polymethoxylated flavones (PFMs) decrease blood serum levels of apoprotein B, the structural protein of LDL cholesterol (Manthey J A. 2004 J. Agric. Food. Chem 52, 7586-7592).

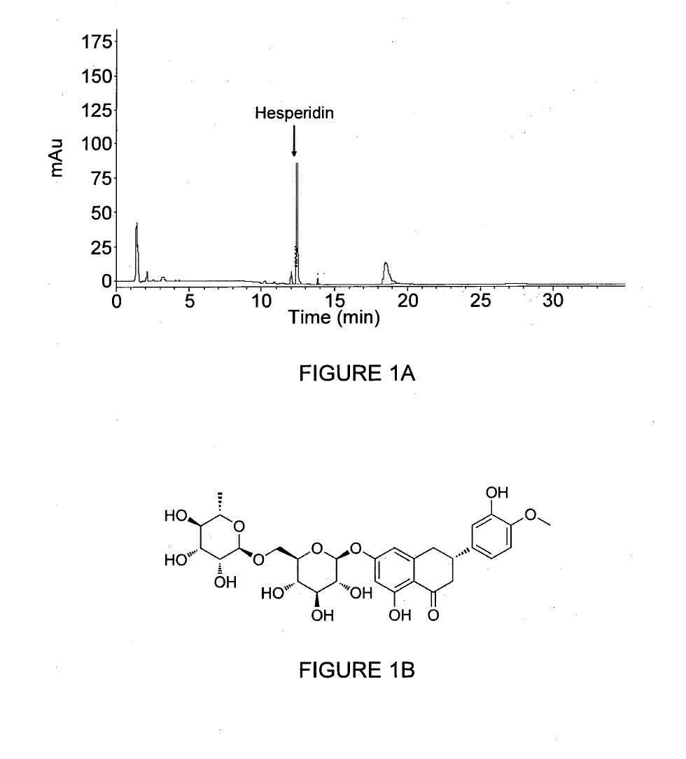

As exemplified hereinbelow, the total polyphenols and flavonoids content within the dry flakes of citrus pulp was 6,920±418 and 4,120±342 mg/100 g dry weight (DW) respectively in in vitro studies, with hesperidin being the major flavonoid with a content of 3,500±240 mg/100 g DW, 85% of the entire total flavonoids fraction (FIG. 1). Moreover, the drying process of the citrus pulp residues to obtain the dry fiber flaxes did not affect the content of the bioactive compounds.

As further exemplified hereinbelow several assays were employed for measuring the antioxidant capacity of citrus flakes of the compositions of the present invention to establish their potential beneficial effects. The results of the total antioxidative capacity as measured by oxygen radical absorbance capacity (ORAC) and trolox equivalent antioxidative capacity (TEAC), as well as the ability to scavenge metal radicals as measured by the ferric reducing ability of plasma (FRAP) assay show that the citrus flakes of the compositions of the present invention have significant anti oxidant activity as compared to known compounds.

Among the naturally occurring citrus flavonoids, hesperidin may be associated with potential health benefits, such as prevention of arteriosclerosis progression. Hesperidin also regulates hepatic cholesterol synthesis by inhibiting the activity of HMG-CoA reductase (Song-Aae B. et al. 1999. J. Nutr. 129, 1182-1185).

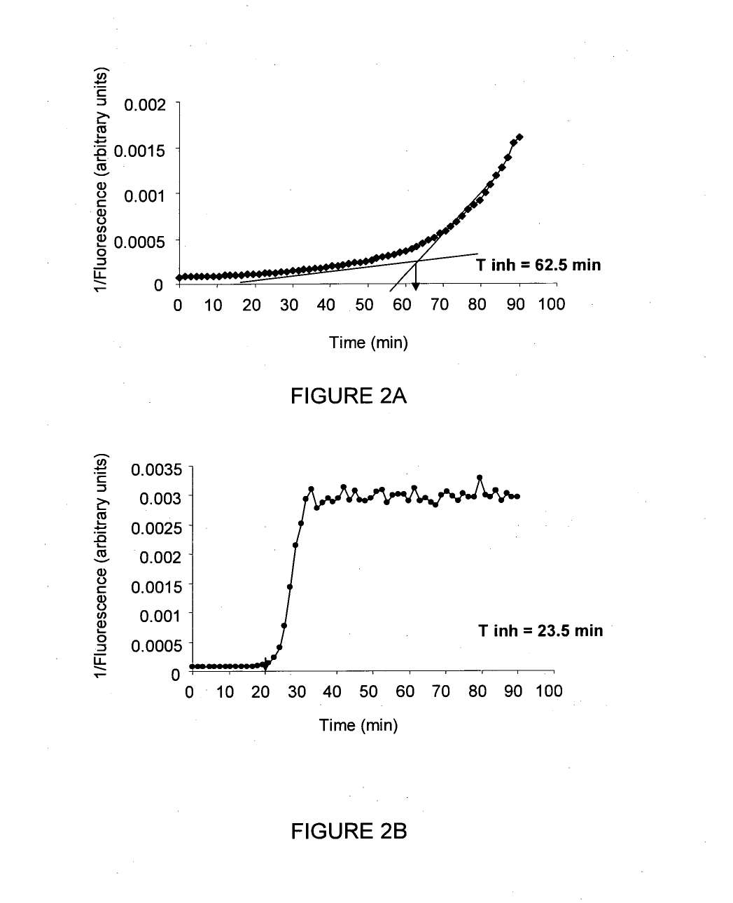

The antioxidant capacity of hesperidin was measured compared to the known anti-oxidant trolox. The antioxidative capacity was represented by T inhibition (T inh). T inh for 10 μM hesperidin or trolox in ORAC system was found to be 62.5 and 23.5 min respectively (FIG. 2). These results demonstrate that hesperidin has a better scavenging activity towards peroxyl radicals, shown by a longer inhibition period, compared to trolox. These results are in agreement with a recent study of Kalpana's and co-workers, showing that hesperidin has a higher antioxidant capacity compared to trolox in protecting pBR322 DNA and red blood cells (RBC) membrane systems when oxidative damage was induced by H₂O₂ (Kalpana K B et al., 2009 Mol Cell Biochem 2009. 323, 21-29).

Hesperidin has lowering effects on serum triglycerides, cholesterol and LDL. Moreover, hesperidin significantly increased HDL levels in hypercholesterolemic humans and rats and elevated the HDL to LDL ratio (Monforte M T. et al., 1995. Farmaco. 50, 595-599; Kurowska E M. et al., 2000. Am. J. Clin. Nutr. 72, 1095-1100). In addition, an inhibiting effect on LDL oxidation was observed. Without wishing to be bound by any specific theory or mechanism of action, the hesperidin's protective effect may be attributed to its direct antioxidant effect as a radical scavenger and to its indirect effect on the endogenic antioxidative system. In this regard it was shown that hesperidin increases the activity of endogenic antioxidative enzymes such as catalase and superoxide dismutase (SOD) (Wilmsen P K. et al. 2005. J Agric Food Chem 53, 4757-4761).

It has been reported that citrus extract comprises polyphenols that show strong lipolytic effect on human body fat adipocytes. The lipolytic activity is mediated by inhibition of cAMP-phosphodiesterase (cAMP-PDE) or of lipase activity.

The present invention now shows a high linear correlation between the anti-oxidative activity and the hesperidin concentration in the orange flakes used in the compositions of the invention (R²=0.957). Without wishing to be bound by any specific theory or mechanism of action, the flavonoids and polyphenols present in the citrus-derived fiber fraction of the composition of the present invention, particularly hesperidin, may contribute to the regulation of the serum lipid and glucose level by regulating the hepatic cholesterol synthesis and/or increasing the lipolysis rate, and/or elevating leptin levels and/or scavenging free radicals and/or increasing the activity of endogenous antioxidative systems.

Flavoring Agents

The present invention now discloses that certain flavors added to the compositions of the present invention overcome the bitterness and tannin-like astringent taste imposed by the citrus-derived flakes serving as the active ingredient of the compositions. According to certain embodiment, the flavoring agent is selected from the group consisting of sesame, sesame paste, date fruit and products thereof, chocolate, coconut, peanuts, peanuts-butter, raisins, apricot, peach, cranberries, other berries such as blueberries, banana and any combination thereof.

Unexpectedly, using sesame or dates as a flavoring agent was found to be most effective in improving the hitherto unpalatable taste of equivalent compositions. Use of sesame and/or dates as a flavoring agent is highly advantageous according to the teachings of the present invention, as, in addition to its taste, seeds of sesame and date fruit, as well as date honey and date paste have further nutritional values. The sesame seeds are rich in iron, manganese, copper, and calcium, and contain vitamin B1 (thiamine) and vitamin E (tocopherol). They contain lignans, including unique content of sesamin, which are phytoestrogens with antioxidant and anti-cancer properties. Among edible oils from six plants, sesame oil had the highest antioxidant content. Sesame seeds also contain phytosterols. This ant-oxidative function is additive to the anti-oxidative activity of the flavonoids present in the citrus fibers, and thus the dietary composition of the present invention has effective anti-oxidative activity, particularly contributing to the inhibition of the deleterious LDL oxidation.

Iron is essential to nearly all known organisms. In cells, iron is generally stored in the centre of metalloproteins. Meat provides the main source for iron, such that vegetarian diet is often iron deficient, and may lead to iron deficiency anemia. Sesame seeds and paste are considered as an excellent source for bioavailable iron that can be readily absorbed and utilized by the mammalian body.

Dates are source for soluble fibers, potassium and several vitamins, particularly of B-complex vitamins—thiamin, riboflavin, niacin, vitamin B-6 and pantothenic acid. Dates are also known as a good source for iron. Typical nutritional value of dates for 100 g is: energy: 259 Kcal; total carbohydrates: 62.5 g; total fiber: 8.5 g; protein: 1.6 g; potassium: 650 mg; total fats: 300 mg; sodium: 4 mg; iron: 2 mg.

It is to be explicitly understood that further flavoring agents may be added to the compositions of the present invention, including, for example rum, vanilla, cinnamon, peanut butter and the like.

Further Ingredients

The compositions of the present invention are preferably formulated in a unit dosage form, for example as bars of from about 25-50 g. Comminuted fruit may optionally be added to the compositions mainly to further improve its mechanical properties for packaging and transport. Various fruit may be used, with fruit providing the desired structural enhancing properties as well as comprising additional active ingredient such as anti-oxidants being preferable. As an example, apple puree may be used for this purpose.

It is to be explicitly understood that additional structure-enhancing compounds may be used, including, but not limited to, fructose, various resins, oats and puffed rice.

Many scientific publications have strongly suggested that regular consumption of significant amounts of polyunsaturated fatty acids can provide important health benefits. In recent years, omega-3 polyunsaturated fatty acids have gained particular attention. Hence, many efforts have been made by the industry to develop food products and nutritional preparations that contain appreciable amounts of omega-3 polyunsaturated fatty acids. According to certain embodiments, the composition of the present invention further comprises at least one polyunsaturated fat selected from the group consisting of omega-3 fatty acid and omega-6 fatty acid. According to one embodiment, the polyunsaturated fat is from a plant source. According to another embodiment, the polyunsaturated fat is from a fish source.

According to further embodiments, the composition further comprises at least one preservative. According to typical embodiments, the preservative is selected from the group consisting of propylene glycol/glycerol and citric acids.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Materials and Methods

### Reagents

Folin-Ciocalteu reagent, Na₂CO₃, Gallic acid, NaNO₂, NaOH, AlCl₃×6H₂O, (+)-Catechin, hesperidin, 6-hydroxy-2,5,7,8,-tetramethylchroman-2-carboxylic acid (Trolox), Fluorescein (FL), 2,2′-azobis-2-methyl-propanimidamide (AAPH), Potassium per-sulfate (K₂O₈S₂) and 2,4,6-Tris(2-pyridyl)-s-triazine (TPTZ), were purchased from Sigma-Aldrich (Rehovot, Israel). Acetonitrile (HPLC grade) was purchased from Biolab (Jerusalem, Israel). Redistilled water was filtered through 0.45 μm membrane before use.

### Fiber Source

Orange (Valencia variety) whole fruit or fruit after the juice was squeezed (orange skin) were purchased from Prigat industry (Kibutz Givat-Haim, Israel). The ground orange fruit or skins were applied to dehydration drums and dehydrated under temperature and time conditions required to achieve the desired dryness of about 5%-10% water content. Typically, temperature of 110° C. for 1 min. resulted in final water content below 10%. The procedure was taken place in Tavlinei Ha-Galil (Afula, Israel). As a result orange flakes were obtained.

### Citrus Flake Extraction

The citrus flakes were extracted with 70% methanol. The slurry was centrifuged at 10,000 rpm and the supernatant was decanted. The extraction procedure was repeated 4 times. The supernatant fluids from all extractions (4 times) were combined and evaporated in vacuum at 30° C. to a final volume of 2.0 ml. The concentrated extract (2.0 ml) was filtered through 0.45 μm membrane. The concentrated extract was taken for analysis of the concentration of polyphenols, flavonoids and flavone glycoside and evaluation of antioxidant capacity.

### Determination of Total Polyphenols Contents in Fresh and Dry Orange Peel

Total content of polyphenols was determined by the Folin-Ciocalteu colorimetric methods (Singleton L. et al. 1999. Methods Enzymol 299, 152-178). The appropriate dilution of extracts was oxidized with the Folin-Ciocalteu reagent, and the reaction was neutralized with Na₂CO₃ solution. Absorbance was measured at 750 nm against the prepared methanol blank. Total citrus flakes polyphenolic extract was expressed on a fresh or dry weight basis as mg of gallic acid equivalents/100 g fresh or dry weight±SE for at least three replications.

### Determination of Total Flavonoids Contents in Fresh and Dry Orange Peel

Total flavonoids content was determined colorimetrically as described previously (Jia Z S. et al. 1994. Food Chem 64, 555-559). Briefly, appropriate dilutions of sample extracts were reacted with NaNO₂ solution, followed by reaction with AlCl₃×6H₂O to form a flavonoid-aluminum complex. Subsequently, NaOH solution was added to the mixture. Absorbance was measured against a prepared blank at 510 nm. The flavonoids content was determined by a (+)-Catechin standard curve and expressed as mean of mg (+)-Catechin equivalents/100 g fresh or dry weight±SE for at least three replications.

### Determination of Hesperidin Content in Fresh and Dry Orange Peel

Reversed-phase liquid chromatography (RP-LC) was used to determine the content of hesperidin, the major flavonoid in orange peel. The HPLC system (Thermo separation products, Riviera Beach, Fla., U.S.A) consisted of an auto-sampler (AS3000), an injector (100 μl), a column oven (30° C.), and a pump (P3000) and a diode-array detector (UV6000). A reversed-phase (RP) C₁₈ column (250 mm×3.2 mm, Phenomeniex, spherisorb 5u 00S-2), was employed. Elution was preformed by a linear gradient with the mobile phase consisting 1.5% (v/v) acetic acid (solvent A) and Acetonitrile (solvent B) at a solvent rate of 0.8 ml/min. The solvents were mixed using a linear gradient composition: 90% A and 10% B for 5 min. then 5% A, 95% B for 20 min, followed by 10% A and 90% B for 10 min. Hesperidin was monitored at 280 nm. The injection volume was 20 μL. Hesperidin was identified according to the spectra derived from photodiode array detection between 200-750 nm, in comparison with authentic standards and by spiking with a standard of hesperidin.

Quantification of hesperidin was determined by RP-LC using hesperidin equilibration curve and expressed as mean of mg hesperidin/100 g fresh crush or dry orange flakes±SE for at least three replications. All samples were filtered through 0.45 μm syringe filter.

### Antioxidant Activity Determinations

A. Oxygen Radical Absorbance Capacity (ORAC) Assay

ORAC assay for fresh orange crush and dry flake extracts was carried out following the modified procedure of the method previously described by Prior et al (Prior R L. et al. 2003. J Agric Food Chem 5, 3273-3279). This assay measures the ability of antioxidant components in test materials to inhibit the decline in disodium fluorescein (FL) fluorescence that is induced by the peroxyl radical generator 2′,2′-Azobis (2-amidinopropane) dihydrochloride (AAPH). The reaction was obtained on fluorescent plate reader (Synergy HT, Bio-Tek Instruments, Winooski, Vt., USA). The mixture contained in the final assay mixture 200 μL of total volume, FL, 120 μL, 70 final concentration and AAPH, 60 μL 32 mM final concentration. All reagents were prepared with 75 mM phosphate buffer, pH 7.4. FL, phosphate buffer and samples were pre-incubated at 37° C. for 15 min. The reaction was started by the addition of AAPH. Fluorescence was measured and recorded every 1.5 min at excitation length of 485 nm and emission length of 535 nm, until the fluorescence of the last reading declined to a value of less than or equal to 5% of the first reading. Phosphate buffer was used as a blank and 2.5-40 μM trolox were used as positive control standards. The final ORAC values were calculated by using a regression equation between the trolox concentration and the net area under the FL decay curve and were expressed as trolox equivalents (TE) as μmole per 100 g of fresh weight (FW) or dry weight (DW).

B. Ferric Reducing Ability of Plasma (FRAP) Assay

The FRAP assay was done according to the method of Benzie and Strain (Benzie I F E. et al. 1999 Anal Biochem 239, 70-76) with some modifications. The stock solution included 300 mM acetate buffer (3.1 g CH₃COONa.3H₂0 and 16 ml CH₃COOH) pH 3.6, mM 2,4,6-tripyridyl-s-triazine (TPTZ) solution in 40 mM HCl, and 20 mM FeCl₃.6H₂0 solution. The fresh working solution was prepared by mixing 25 ml acetate buffer, 2.5 ml TPTZ solution, and 2.5 ml FeCl₃.6H₂0 solution and then warmed at 37° C. before using. Extracts of citrus flakes prepared as described above and of the citrus source material (wet crushed orange fruit) extract according to the same method (150 μl) were allowed to react with 2850 μl of the FRAP solution for 30 min. in dark conditions. Readings of the colored product (ferrous tripyridyltriazine complex) were then taken at 593 nm. Results are expressed in μM TE/100 g fresh fruit or dry flakes.

C. Trolox Equivalent Antioxidant Capacity (TEAC)

TEAC assay for fresh orange and dry flakes extracts was carried out following the procedures previously described by Arnao et al. (Arnao M B. et al. 2001 Food Chem 73, 239-244) with some modifications. 2,2′-azino-bis(3-ethylbenzothiazoline-6-sulfonic acid (ABTS) was dissolved in water to a 7 mM concentration. ABTS⁺ radical cation was produced by reacting ABTS stock solution with 2.45 mM potassium persulphate (final concentration) and allowing the mixture to stand for 12-16 hr at room temperature in the dark before use. The solution was then diluted by mixing 1 ml ABTS⁺ solution with 60 ml ethanol to obtain an absorbance of 0.7±0.02 at 734 nm. Fruits extracts (150 μl) were allowed to react with 2850 μl of ABTS⁺ solution for 6 min in dark condition. Then the absorbance was taken at 734 nm. Results are expressed in μM trolox equivalents (TE)/100 g fresh or dry fruit.

### Determination of Dietary Fiber

Samples of source material (wet crushed orange fruit) or citrus flake were analyzed for soluble and insoluble dietary fibers fractions according to the Association of official agricultural chemists (AOAC) method 991.43. The method is based on an enzymatic-gravimetric procedure. Briefly, samples containing high content of sugar were extracted with 85% ethanol to remove most of the sugars. Residues were suspended with MES-TRIS buffer and digested sequentially with heat stable α-amylase at 95-100° C., protease at 60° C. and amyloglucosidase at 60° C. After digestion, the solutions were filtered through tarred fritted glass crucibles. Crucibles containing insoluble dietary fiber were rinsed with dilute alcohol followed by acetone, and have dried in the oven for overnight in a 105° C. Filtrates and washing were mixed with 4× volume of 95% ethanol to precipitate materials that were soluble after digestion. After one hour, precipitates were filtered through tarred fritted glass crucibles. One of each set of duplicate insoluble fiber residues and soluble fiber residues was burned to ash in a muffle furnace at 525° C. for 5 h. Another set of residues was used to determine protein as kjeldahl nitrogen×6.25. Soluble or insoluble dietary fiber residues (% original sample weight) minus % ash and minus % crude protein found in the residues were taken to be the values for the respectively dietary fiber fraction. Total dietary fiber was calculated as the sum of soluble and insoluble dietary fibers (Li W et al. J Food Compos Anal 15, 715-723).

### Example 1

### Preparation of the Composition

Typically, the composition was prepared as followed:
1. Sweeteners (selected from brown sugar, white sugar, honey, isomalt, putted dates and/or another polyols such as sorbitol, maltitol, xylitol, erythritol, lactitol) are heated to melting. Optionally, apple puree is added. The mixture is heated to 115° C. to obtain syrup. Heating is stopped once the temperature reaches 115° C.
2. Citrus flakes are added to the syrup and, stirred until completely covered with the syrup.
3. Flavoring agents, preservatives and additional structure enhancing ingredient (except of puffed rice) are added and the mixture is well stirred.
4. Puffed rice is added and the mixture is further stirred.
5. The resulting composition is spread and cooled to room temperature. The thickness of the layer depends on the desired size of the bar.
6. The layer is cut to bars of about 25 g to 50 g.

The content of total, soluble and insoluble dietary fibers in dry orange flakes were 53.1±4.9, 21.9±2.4 and 31.2±3.1 g/100 g DW respectively.

Examples of several differently flavored bar content are provided in tables 1-6 below.

TABLE 1
Sesame-flavored bar

| | Percentage |
|---|---|
| Ingredient | (%) |
| Water | 0 |
| Brown Sugar | 15 |
| Honey | 9 |
| Apple Pure | 10 |
| Orange Flakes | 12 |
| Oats | 10 |
| Tahini | 11.5 |
| Roasted Sesame | 7.5 |
| Sunflower Seeds | 5 |
| Isomalt | 15 |
| Puffed Rice | 5 |

TABLE 2
Coconut-flavored bar

| Ingredient | Percentage (%) |
|---|---|
| Water | 0.0 |
| Brown Sugar | 15.0 |
| Honey | 10.0 |
| Apple Pure | 10.0 |
| Orange Flakes | 12.0 |
| Oats | 10.0 |
| Puffed Rice | 9.0 |
| Isomalt | 14.0 |
| Coconut Flakes | 15.0 |
| Cocoa Powder | 3.0 |
| Fats (Canola, Palm, lecithin) | 2.0 |

TABLE 3
Chocolate-flavored bar

| Ingredient | Percentage (%) |
|---|---|
| Water | 10.0 |
| Brown Sugar | 7.0 |
| Honey | 7.0 |
| Apple Pure | 10.0 |
| Orange Flakes | 10.0 |
| Oats | 10.0 |
| Puffed Rice | 2.0 |
| Fats (Canola, Palm, lecithin) | 10.0 |
| Chocolate | 12.0 |
| Isomalt | 9.0 |
| Walnuts | 13.0 |

TABLE 4
Date-flavored bar

| Ingredient | Percentage (%) |
|---|---|
| Water | 5.1 |
| Natural date syrup | 14.0 |
| Oat | 6.7 |
| Dates paste | 10.4 |
| Dried pitted dates | 9.6 |
| Puffed rice | 8.0 |
| Corn syrup (glucose syrup 85/42) | 7.6 |
| Hazelnuts | 7.0 |
| Walnuts | 7.0 |
| White sugar | 7.0 |
| Dried orange flakes | 10 |
| Almonds | 6.0 |
| Phytosterols | 1.6 |
| Cinnamon | 0.1 |

TABLE 5
Cranberry-flavored bar

| Ingredient | Percentage (%) |
|---|---|
| Natural date syrup | 14.0 |
| Water | 5.1 |
| Oat | 6.7 |
| Dates paste | 10.4 |
| Dried Cranberries | 9.6 |
| Puffed rice | 8.0 |
| Corn syrup (glucose syrup | 7.6 |
| 85/42) | |
| Hazelnuts | 7.0 |
| Walnuts | 7.0 |
| White sugar | 7.0 |
| Dried orange flakes | 10. |
| Almonds | 6.0 |
| Phytosterols | 1.6 |

TABLE 6
Date-orange bar

| Ingredient | Percentage (%) |
|---|---|
| Water | 5.0 |
| White sugar | 8.9 |
| (sucrose) | |
| Natural date syrup | 17.8 |
| Ground pitted dates | 17.8 |
| Dried orange flakes | 5.7 |
| Fine ground oat | 11.9 |
| Puffed rice | 13.3 |
| Almonds | 5.9 |
| Hazel nuts | 8.9 |
| Cinnamon | 0.5 |
| Natural palm oil | 4.0 |
| Salt | 0.3 |

### Example 2

### Concentration of Bioactive Compounds in Fresh Orange Crush and in Dry Flakes

The concentrations of the bioactive compounds are given in Table 7 hereinbelow. The total polyphenols, flavonoids and hesperidin contents in dry orange flakes were 6,920±418, 4,120±342 and 3,500±240 mg/100 g DW respectively. The total polyphenols, flavonoids and hesperidin contents in fresh orange crush were 672±49 mg/100 g FW of gallic acid equivalents, 397±30 mg/100 g FW and 342±25 mg/100 g FW respectively. Total polyphenols were obtained on the basis of gallic acid equivalents. Total flavonoids were obtained on the basis of (+) catechin equivalents.

TABLE 7
Total content of polyphenols, flavonoids and hesperidin (mg/100 g DW
or FW) in orange fractions before and after drying.

| | | Total flavonoids | |
|---|---|---|---|
| | Total polyphenols | (mg of (+) | Hesperidin |
| Fraction | (mg of GAE/100 g) | catechin/100 g) | (mg/100 g) |
| Dried orange | 6,920 ± 418 | 4,120 ± 342 | 3500 ± 240 |
| Flakes (a) | | | |
| Fresh orange | 672 ± 49 | 397 ± 30 | 342 ± 25 |
| crush (b) | | | |

(a): mg/100 g DW (Dry weight);
(b): mg/100 g FW (Fresh Weight)

### Example 3

### Antioxidant Activity

The results of the measurement of ORAC^{(a)}, FRAP^{(b)} and TEAC^{(c)} are summarized in Table 8 hereinbelow. As can be seen in this table, the ORAC value in the dry flakes was 5,408.6±252 μmole of TE/100 g DW and for fresh crush 518.8±36 μmole of TE/100 g FW. The FRAP value in the dry flakes was 230.6±21.2 mole of TE/100 g DW and 21.9±1.8 for the fresh crush. TEAC value in the flakes and in the fresh crush were 2,419.7±234.7 TE/100 g DW and 224.2±20.8 TE/100 g FW respectively.

TABLE 8
Antioxidant capacity of 70% methanol fraction
in orange flakes and in orange crush

| | ORAC^{(a)} (μmole | FRAP^{(b)} | TEAC^{(c)} |
|---|---|---|---|
| Fraction | of TE/100 g) | (μmole of TE/100 g) | (μmoleTE/100 g) |
| Dried orange | 5,408.6 ± 252.3 | 230.6 ± 21.2 | 2,419.7 ± 234.7 |
| flakes (d) | | | |
| Fresh orange | 518.8 ± 36.4 | 21.9 ± 1.8 | 224.2 ± 20.8 |
| crush (e) | | | |

^{(a)}ORAC, oxygen radical absorbing capacity.
^{(b)}FRAP, ferric reducing antioxidant capacity.
^{(c)}TEAC, Trolox equivalent antioxidant capacity;
(d) mg/100 g DW;
(e): mg/100 g FW.

### Example 4

### Antioxidant Activity of Hesperidin Compared to Trolox

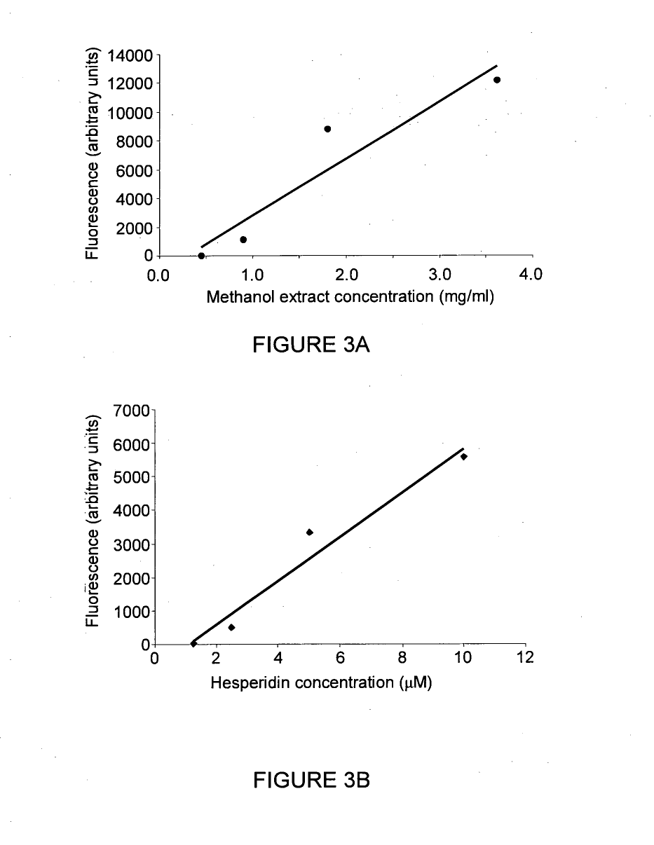

In both compounds the measured inhibition time was directly proportional to the concentration of the antioxidant. In the tasted concentration range 2.5, 5.0, 10.0 μM of trolox the Tᵢₙₕ were 14.6, 20.9, and 25.5 respectively. For comparison Tᵢₙₕ for 2.5, 5.0, 10.0 μM of hesperidin were 26.8, 45.0 and 62.5 min respectively. FIG. 2 demonstrate the antioxidant capacity of hesperidin compared to Trolox by the inhibition time (Tᵢₙₕ). In ORAC assay Tᵢₙₕ measures the ability of antioxidant components in test materials to inhibit the decline in disodium fluorescein (FL) fluorescence that is induced by the peroxyl radical generator 2′,2′-Azobis (2-amidinopropane) dihydrochloride (AAPH). Tᵢₙₕ for 10 μM of hesperidin and 10 μM of trolox were 62.5 and 23.5 min respectively. From these results we can assume that hesperidin can scavenge more peroxyl radicals indicating a longer inhibition period than trolox. A high correlation was demonstrated between concentration and antioxidant capacity for both the complete methanolic extract and hesperidin (R²=0.893 and R²=0.957, FIG. 3A, B respectively). Higher correlation was observed between Hesperidin concentration and its antioxidant capacity.

### Example 5

### Blood Cholesterol Levels in Hypercholesterolemic Patients

### Patients and Treatments

A total of 116 patients known to have unbalanced hypercholesterolemia participated in the study. 62 were women and 51 men, at age range of 40-74 years and 3 children, 2 boys and one girl, ages 7-10 years.

### Dietary Supplements

All participants consumed two snack bars comprising the composition of the invention daily (each snake contains 2.7 g of orange flakes) or parallel amount of orange flakes as a fine powder. The observations lasted 3-6 months for each patient. All patients did not change their lifestyle or eating habits or medications besides adding the citrus bar to their daily nutrition.

### Determination of Blood Cholesterol Levels

All participants in the trial were checked for blood cholesterol levels at time 0, then every two weeks to four weeks. According to accepted procedure, each patient was asked to fast 12-14 h prior to blood sampling.

Plasma serum total cholesterol was measured by an automatic biochemistry analyzer (Roche Diagnostics). High-density lipoprotein cholesterol (HDL-C) was measured directly by an enzymatic in vitro assay that uses poly-ethylen-glycol-modified enzymes in the presence of magnesium sulfate and dextran sulfate to get a selective catalytic activities of lipoprotein fractions (Roche Diagnostics). Low-density lipoprotein cholesterol (LDL-C) was calculated using the Friedewald equation: LDL-C=total cholesterol-(HDL-C+triglycerides/5) (Friedewald W T et al. 1972 Clin Chem 18, 499-502).

### Statistical Analysis

Results were analyzed by JMP IN statistical discovery software using one-way variance analysis (ANOVA). When a significant differences was obtained (P>0.05) the Tukey-Kremer USD test used to compare each pair of means.

### Results

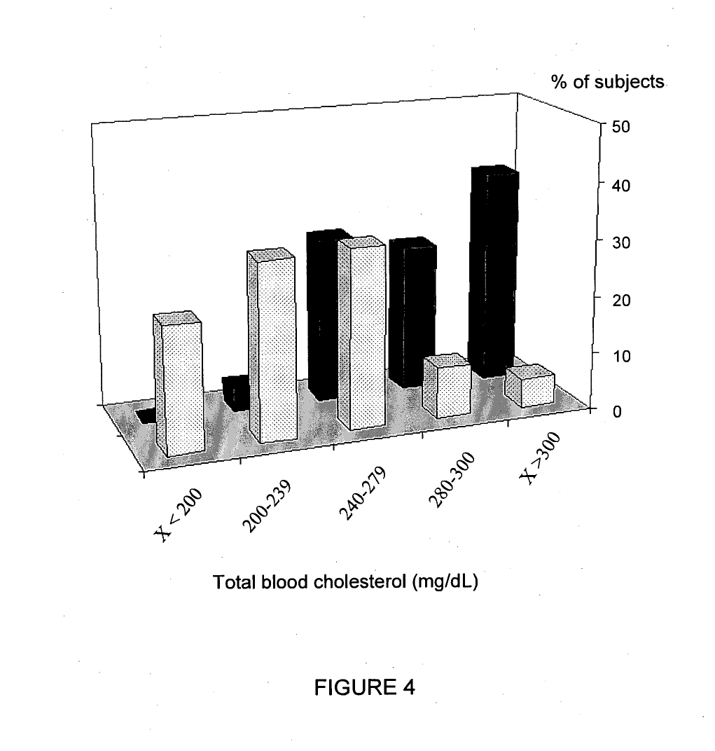

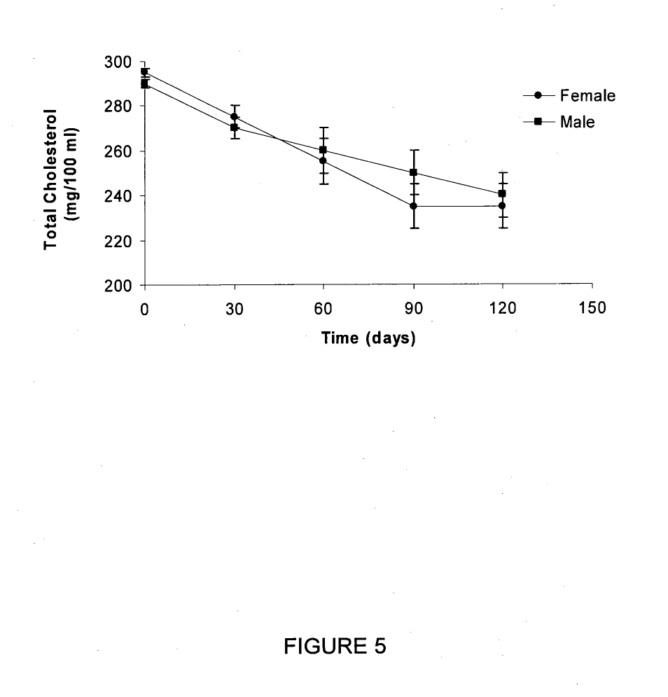

The desirable blood cholesterol level is less than 200 mg/100 ml. None of the patients participating in the study had the desirable concentration at the beginning of the study. 111 from 116 subjects (96%) began the treatment with blood cholesterol level higher than 240 mg cholesterol/100 ml, of which 38% had above 300 and 27% had between 280-300 mg/100 ml. At the end of treatment only 45% of the patients had blood cholesterol level higher than 240 mg/ml (reduction of 51%), of which 10% of had a cholesterol level of between 280-300 (reduction of 17% compare to the beginning) and 13% had between 260-279 (reduction of 6%). 17% of the patients had blood cholesterol levels between 240-259 at the end of treatment (elevation of 5%), 15% of the patients had between 220-239 mg cholesterol/100 ml (elevation of 12%), 17% of the subjects had 200-219 mg/100 ml (elevation of 17%) and 22% had bellow 200 mg/100 ml (elevation of 22%) at the end of the clinical trial (FIG. 4). In addition, Impressive reduction in blood cholesterol levels has been shown both in male and female (FIG. 5).

### Example 6

### HDL to LDL Ratio

One hundred and thirteen (113) subjects suffering from hypercholesterolemia participated in the trial. Each subject consumed two snack bars comprising the composition of the invention daily (each snake comprising 2.7 g of orange flakes).

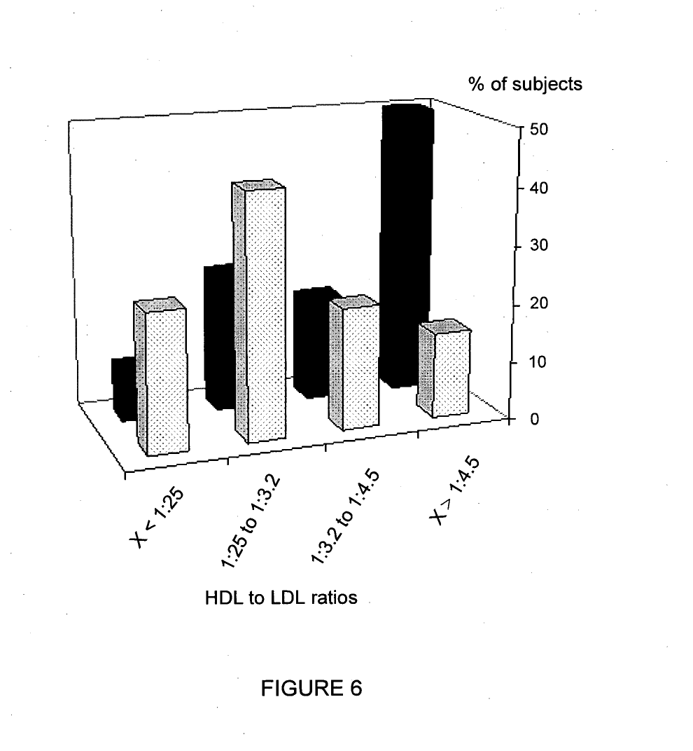

The optimal HDL to LDL ratio is lower than 1:3.2 with the ideal being lower than 1:2.5. At the beginning of the trial, 50% of the 113 participants had HDL to LDL ratio higher than 1:4.5. Only 15% of the patients had such HDL to LDL serum ratio at the end of the trial. At the beginning 9% of the patients had the healthy HDL to LDL serum ratio lower than 1:2.5. At the end 24% of the participants had this serum ratio, i.e. an elevation of 15% (FIG. 6).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. An edible composition effective in reducing the cholesterol blood level comprising a malleable matrix comprising (a) citrus flakes at from about 5% to about 20% by weight of the total weight of the composition, wherein the ratio of the soluble to insoluble fibers is from 1.0:1.5 to 1.5:1.0; and (b) a flavoring agent selected from the group consisting of sesame seeds, sesame paste, chocolate, coconut, banana, dates and any combination thereof; wherein the composition is in a unit dosage form.

2. The composition of claim 1, wherein the ratio of the soluble to insoluble fibers is from 1.0:1.25 to 1.25:1.0.

3. The composition of claim 1, wherein the ratio of the soluble to insoluble fibers is 1:1.

4. The composition of claim 1, wherein the citrus flakes are obtained from a source selected from the group consisting of citrus pulp and crushed orange fruit.

5. (canceled)

6. The composition of claim 1, wherein the citrus flakes comprise polyphenols at from 5% to 10% by weight of the weight of said flakes.

7. The composition of claim 1, wherein the citrus flakes comprise flavonoids at from 2% to 6% by weight of the weight of said flakes.

8. The composition of claim 1, further comprising phytosterols at a concentration of from about 0.5% to about 2.0% by weight of the total weight of the composition.

9. The composition of claim 1, further comprising at least one additional ingredient selected from the group consisting of additional anti-oxidant, a polyunsaturated fat and a combination thereof.

10. The composition of claim 9, wherein the antioxidant is CoQ10 and the polyunsaturated fat selected from the group consisting of omega-3 fatty acid and omega-6 fatty acid.

11. (canceled)

12. (canceled)

13. The composition of claim 1, said composition comprises sesame seeds at from about 3% to about 11% by weight of the total weight of the composition and/or sesame paste at from about 7% to about 15% by weight of the total weight of the composition.

14. The composition of claim 1, said composition comprises date fruit, date fruit product or a combination thereof at a concentration of from about 10% to about 40% by weight of the total weight of the composition.

15. The composition of claim 1, wherein said composition further comprises a carbohydrate selected from the group consisting of fructose, sorbitol, xylitol, glucose, sucrose, dextrose and any combination thereof.

16. The composition of claim 1, said composition further comprises at least one additional dietary ingredient selected from the group consisting of minerals, vitamins, amino acids and hormones.

17. (canceled)

18. The composition of claim 1, wherein the unit dosage form is a nutritional or health bar.

19. (canceled)

20. A method for reducing serum lipid concentration comprising orally administering to a subject in need thereof a therapeutically effective amount of the composition of claim 1.

21. The method of claim 20, wherein the lipid is selected from cholesterol, LDL and triglyceride.

22. The method of claim 21, wherein the serum lipid concentration is reduced by at least 5%.

23. (canceled)

24. The method of claim 15, wherein the composition is in a unit dosage form of a nutritional or health bar and wherein the bar weight is from about 25 to about 50 g.

25. The method of claim 24 wherein the unit dosage form is consumed twice daily.

26. (canceled)

27. (canceled)

28. (canceled)

29. A method for enhancing the feeling of satiety comprising consuming an effective amount of the composition of claim 1.

30. The method of claim 29 wherein the composition is in a unit dosage form of a nutritional or health bar and wherein the bar weight is from about 25 to about 50 g.

31. The method of claim 30, wherein the unit dosage form is consumed at least twice a day.
